(19) United States
(12) Patent Application Publication
(10) Pub. No.: US 2023/0157605 A1
(43) Pub. Date: May 25, 2023

(54) MEASURING MUSCLE LOAD IN ATLETIC ACTIVITIES, AND ASSOCIATED SYSTEMS AND METHODS

(71) Applicant: Strive Tech Inc., Bothell, WA (US)

(72) Inventor: Nikola Mrvaljevic, Bothell, WA (US)

(73) Assignee: Strive Tech Inc., Bothell, WA (US)

(21) Appl. No.: 17/801,170

(22) PCT Filed: Feb. 18, 2021
(86) PCT No.: PCT/US2021/018572

§ 371 Date: Aug. 19, 2022

Related U.S. Application Data
(60) Provisional application No. 62/978,576, filed on Feb. 19, 2020.

(51) Int. Cl. A61B 5/22; A61B 5/397; A61B 5/11; A61B 5/00

(52) U.S. Cl. CPC A61B 5/224; A61B 5/397; A61B 5/1112; A61B 5/6802; A61B 2503/10

(57) ABSTRACT

Measuring muscle load in athletic activities, and associated systems and methods are described herein. In an embodiment, a method for monitoring muscle load of an athlete includes: determining a muscle effort (ME) of the athlete by a wearable electromyography (EMG) sensor, and determining at least one inertial measurement unit (IMU) output of the athlete. The method further includes comparing the ME and the IMU output of the athlete, and, based on comparing, determining a performance of the athlete.

### CROSS-REFERENCE TO RELATED APPLICATION

This application claims the benefit of U.S. Provisional Application No. 62/978,576, filed Feb. 19, 2020, the disclosure of which is incorporated herein by reference in its entirety.

### BACKGROUND

Measuring load in athletic activities today is mostly done using the accelerometer and GPS load measurements. These are usually considered external load measurement. When it comes to internal load measurements, sometimes considered as “effort,” the most common method to date has been heart rate.

Some other conventional technologies evaluate heartbeat rate as a substitute for load exertion of the athlete. However, systems and methods for improved observation and measurement of the power of the athlete during exercise are still needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, the inventive technology combines different measurements that evaluate muscle load of the athlete. Such measurements may include muscle effort, heart effort, GPS, and inertial measurement unit (IMU) output that represent the force exerted by the athlete. In some embodiments, measurements of muscles may also be used to monitor effort and load on the body during athletic activities. Furthermore, data obtained from a global positioning system (GPS) and gyroscopes attached to the athlete may indicate a location, trajectory or orientation of the athlete. When these measurements of the effort of the athlete and force exerted by the athlete are combined, a load of the athlete can be estimated.

Furthermore, an efficiency of the athlete can be measured by observing, for example, how much muscle or heart effort was required to run a certain distance within a given time. For example, one athlete may run a prescribed distance s within a time t using a muscle effort (ME). The other athlete may run the same distance s within 10% longer time t, but with a muscle effort that is 40% less than the ME of the first athlete. Under the above scenario, the second athlete would possess a higher potential for athletic improvement. Another possible conclusion is that the first athlete may be sick or exhausted if his/her ME is close to a maximum that this athlete can exert. Furthermore, an increased muscle effort ME or heart effort (HE) by the first athlete that is not accompanied by a corresponding increase in the acceleration or distance travelled (i.e., power) may indicate a relatively poor technique of the athlete, thus needing an improvement.

Many conventional technologies measure speed, distance and location of the athlete without taking into account a physical size of the athlete. With some embodiments of the present technology, the ME or HE is calibrated per physical size of the athlete for more precise correlation to the power of the athlete. Furthermore, many GPS-based conventional technologies only account for the power expended by the athlete within a horizontal plane. With some embodiments of the present technology, the vertical movements of the athlete, such as during vertical jumps, are also accounted for within the total energy expenditure.

In the context of this application, the determination of load expended by a user is described with reference to the user being an athlete. However, the inventive technology is also applicable to determination of the load expended by, for example, soldiers, workers, couriers, etc., that are equipped with clothing that carries suitable sensors and/or processors described herein.

Analytics systems configured in accordance with various embodiments of the present technology, can address at least some limitations of traditional methods of detecting fatigue and/or monitoring athletic performance. As described below, the system can provide analytics that are real-time, comparative, and predictive in nature. This, in turn, provides the opportunity for improved training outcomes, and earlier intervention and corrective action to reduce the risk of fatigue-related injuries.

Various embodiments of the present technology a real time analytics system incorporating data collected from wearable sensor technology, also referred to as a performance monitor, into an interactive user interface having a receiver, such as a wireless receiver, for sensor data. In different embodiments, the inventive technology may be used for other purposes. For example, the inventive technology may be used for military training or in conjunction with consumer devices.

In some embodiments, the user interface may communicate with a data storage system including a processor implementing machine learning analytics. The interactive user interface may be implemented on a digital platform that analyzes real-time data collected from the wearable sensor technology as the subject exercises or rests, and may compare the collected data with aggregated data collected from additional subjects and subsequently analyzed by a machine learning system. The machine learning analytics may implement predictive models such as likelihood of injury, asymmetric exertion, motion or posture irregularities, etc.

As understood by one of ordinary skill in the art, a “data storage system” as described herein may be a device configured to store data for access by a computing device. An example of a data storage system is a high-speed relational database management system (DBMS) executing on one or more computing devices and being accessible over a high-speed network. However, other suitable storage techniques and/or devices capable of quickly and reliably providing the stored data in response to queries may be used, and the computing device may be accessible locally instead of over a network, or may be provided as a cloud-based service. The data storage system may also include data stored in an organized manner on a computer-readable storage medium.

### DESCRIPTION OF THE DRAWINGS

The foregoing aspects and attendant advantages of the inventive technology will become more readily appreciated as the same become better understood by reference to the following detailed description, when taken in conjunction with the accompanying drawings, wherein:

FIG. 1 is a partially schematic view of athlete's clothing in accordance with the present disclosure.

FIG. 2 illustrates an inner side of athlete's pants in accordance with the present disclosure.

FIG. 3 illustrates an outer side of athlete's pants in accordance with the present disclosure.

FIG. 4 is a schematic view of a performance monitoring system in accordance with the present disclosure.

FIG. 5 is a flowchart of a method of assessing athletic performance in accordance with the present disclosure.

FIG. 6 is a graph of muscle load of athlete in accordance with the present disclosure.

### DETAILED DESCRIPTION

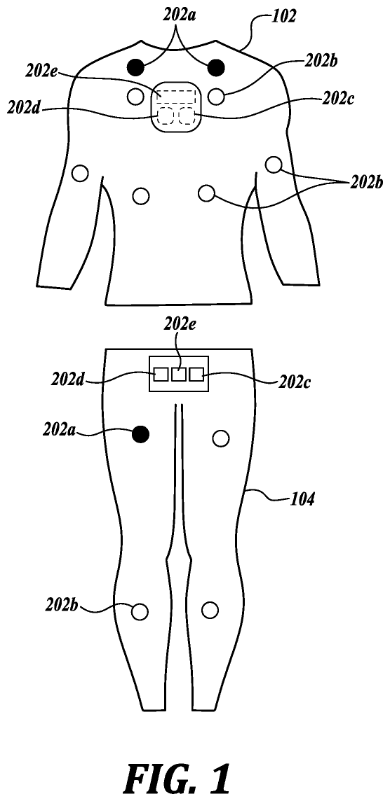

FIG. 1 is a partially schematic view of athlete's clothing in accordance with the present disclosure. In the illustrated embodiment, the athlete's clothing includes upper clothing 102 (e.g., a shirt) and lower clothing 104 (e.g., pants). However, in other embodiments the required sensors and electronics may be carried by the lower clothing 102 only or the upper clothing 104 only.

The athlete's clothing 102/104 can carry various sensors like, for example, electrocardiogram (ECG) sensors 202a, electromyography (EMG) sensors 202b, an orientation sensor 202c (e.g., a gyroscope), an acceleration sensor 202d (e.g., an accelerometer), and a global positioning (GPS) locator 202e. These sensors may be distributed over various locations on the athlete's clothing. The sensors 202a-202e can be operationally connected to a controller using thin, resilient flexible wires and/or conductive thread woven into the clothing 102/104.

The ECG and EMG sensors 202a and 202b may include dry-surface electrodes distributed throughout the athlete's clothing 102/104 to make necessary skin contact beneath the clothing along predetermined locations of the body. In some embodiments, the ECG and EMG sensors 202a and 202b can include an optical detector, such an optical sensor for measuring heart rate or muscle contraction. The fit of the clothing may be sufficiently tight to provide continuous skin contact with the individual sensors 202a-202e, allowing for accurate readings, while still maintaining a high-level of comfort, comparable to that of traditional compression fit shirts, pants, and similar clothing. In various embodiments, the clothing 102/104 can be made from compressive fit materials, such as polyester and other materials (e.g., Elastaine) for increased comfort and functionality. In some embodiments, the sensors 202a-202e can have sufficient durability and water-resistance so that they can be washed with the clothing 102/104 in a washing machine without causing damage.

The EMG sensors 202b can be positioned adjacent to targeted muscle groups, such as the large muscle groups of the pectoralis major, rectus abdominis, quadriceps femoris, biceps, triceps, deltoids, gastrocnemius, hamstring, and latissimus dorsi. The EMG sensors 202b can also be coupled to floating ground near the athlete's waist or hip.

The orientation and accelerations sensors 202c and 202d may be disposed at a central position between the athlete's shoulders and upper back region. In some embodiments, the central, upper back region can be an optimal location for placement of the orientation and acceleration sensors 202c and 202d, because of the relatively small amount of muscle tissue in this region of the body, which prevents muscle movement from interfering with the accuracy of the orientation and acceleration readings. In other embodiments, the orientation sensor 202c and/or the acceleration sensor 202d can be positioned centrally on the user's chest, tail-bone, or other suitable locations of the body. An example of a suitable location is a belt region (waste) of the lower clothing 104. In some embodiments, multiple acceleration sensors and/or orientation sensors may be used for detecting acceleration and/or orientation of athlete's torso or one or more of the athlete's limbs. The GPS sensor 202e may be attached to a part of the athlete's clothing that is representative of the location of the body of the athlete (e.g., for example a chest of the athlete or a thigh of the athlete).

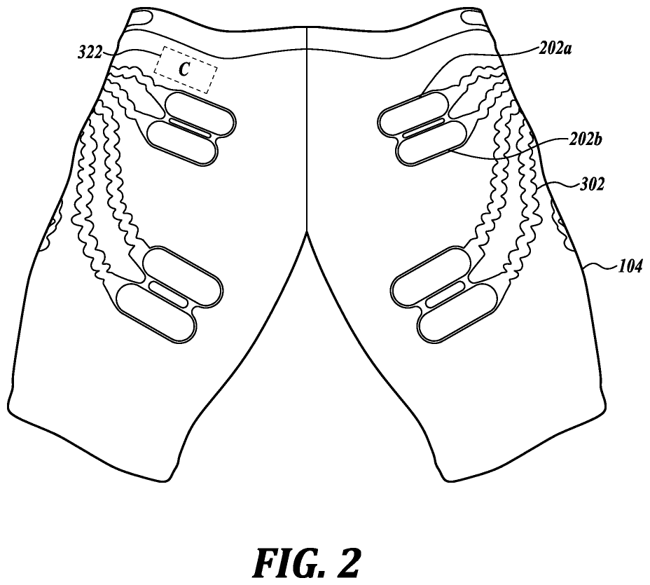

FIG. 2 illustrates an inner side of athlete's pants 104 in accordance with the present disclosure. In the illustrated embodiment, the athlete's pants 104 carry the ECG sensors 202a and the EMG sensors 203b. The sensors are connected through wiring 302 with appropriate controllers, for example a controller 322.

The controller 322 can be embedded within the athlete's clothing, such as the pants 104. In other embodiments, the controller 322 can be inserted into a pocket in the user's clothing and/or attached using Velcro, snap, snap-fit buttons, zippers, etc. In some embodiments, the controller 322 can be removable from the clothing 102/104, such as for charging the controller. In other embodiments, the controller 322 can be permanently installed in the athlete's clothing.

In one aspect of this embodiment, the use of a single orientation sensor and a single acceleration sensor can reduce computational complexity of the various analytics produced by the system. In particular, a reduced set of orientation and acceleration data may be sufficient for detecting various indicators of fatigue and other performance characteristics in conjunction with the other real-time data. In other embodiments, however, the performance of the athlete can be monitored through multiple acceleration sensors and/or orientation sensors, such as for detecting acceleration and/or orientation of one or more of the athlete's limbs.

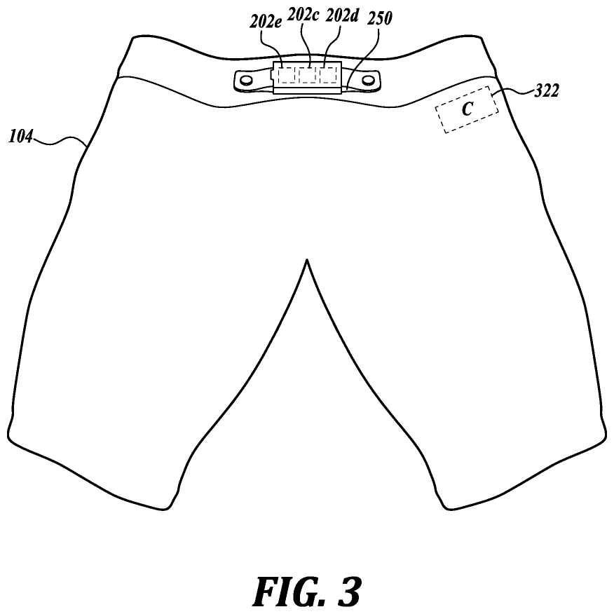

FIG. 3 illustrates an outer side of athlete's pants 104 in accordance with the present disclosure. In the illustrated embodiment, the athlete's pants 104 carry a pouch 250 that, in turn, carry one or more orientation sensors 202c and one or more acceleration sensors 202b. In some embodiments, a relatively central location of the pouch 250 may improve sensing of the acceleration of the body during, for example, jumps of the athlete, while still being able to sense horizontal movements of the athlete. Furthermore, such central location of the pouch 250 may be less sensitive to the spurious orientation signals (e.g., caused by the limbs of the athlete), thus enabling the orientation sensor 202c to sense the orientation that is more representative of the entire body of the athlete. In operation, the orientation sensors 202c and acceleration sensors 202b may communicate with the controller C.

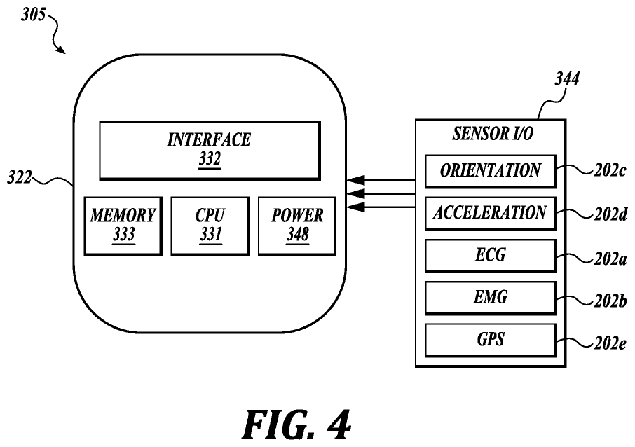

FIG. 4 is a schematic view of a performance monitoring system 305 (also referred to as a performance monitor) in accordance with the present disclosure. In operation, the sensors 202a-202e communicate with the controller 322 wirelessly or through electrical wires. Data from the sensors are received by an interface 332, which may be wireless or wired interface. In different embodiments, the controller 322 may include a memory 333, a CPU 331, and power source 348.

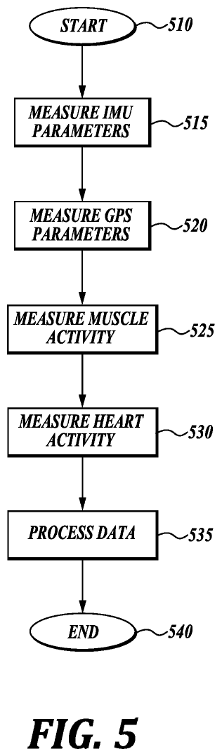

FIG. 5 is a flowchart of a method of assessing athletic performance in accordance with the present disclosure. The method may start in block 500. In block 515, different IMU parameters (e.g., acceleration, rotation of the body) are measured. In block 520, GPS parameters are measured (e.g., location of the athlete). In block 525, the muscle activity of the athlete is measured. In some embodiments, muscle load can be expressed as a combined loading of different groups of muscles. An example of such muscle load is shown in eq. 1 below:

$$Muscle Load = {\sum }_{i = 1}^{n} \sqrt{{LQ}_{i}^{2} + {RQ}_{i}^{2} + {LH}_{i}^{2} + {RH}_{i}^{2} + {LG}_{i}^{2} + {RG}_{i}^{2}} \qquad Eq . ( 1 )$$

where LQ and RQ represent muscle load of the left and right quad muscles, respectively. LH and RH represent muscle load of the left and right hamstring muscles, respectively, and LG and RG represent muscle load of the left and right glute muscles, respectively.

In block 530, the heart activity of the athlete is measured. Thus-acquired data may be processed in block 535. As explained above, the processing may include determination of the power, energy, efficiency and/or fatigue of the athlete. The method may end in block 540.

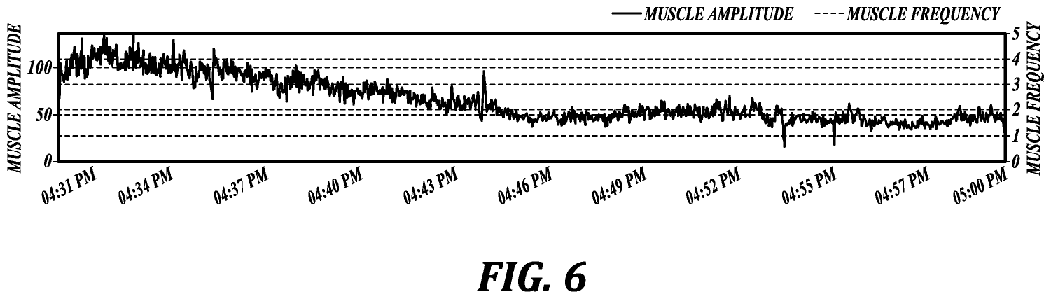

FIG. 6 is a graph of measured muscle load of athlete in accordance with the present disclosure. The horizontal axis of the graph shows time. The vertical axis of the graph shows muscle amplitude and muscle frequency, as indicated in the graph. In particular, the power measurements were obtained using the IMU measurements (e.g., acceleration, GPS), while the muscle load measurements were obtained using the EMG sensors.

For the measurements shown in FIG. 6, the pouch 250 was located on the belt buckle area. The muscle exertion to move the body forward was measured with EMG sensors. In combination, these measurements provide understanding (using actual muscle reading) of the muscle load used by a user to produce a certain effort (i.e., to move the body in a certain direction for a given distance).

While various advantages associated with some embodiments of the disclosure have been described above, in the claims, other embodiments may also exhibit such advantages, and not all embodiments need necessarily exhibit such advantages to fall within the scope of the invention. For example, while various embodiments are described in the context of an athlete (e.g., a professional or collegiate athlete), in some embodiments users of the system can include novice or intermediate users, such as users, trainers, and coaches associated with a high school sports team, an athletic center, a professional gym, etc. In other embodiments, the users may be military personnel, workers, couriers, or other personnel whose performance is measured. Accordingly, the disclosure is not limited, except as by the appended claims.

What is claimed is:

1. A method for monitoring muscle load of an athlete, comprising:
determining a muscle effort (ME) of the athlete by a wearable electromyography (EMG) sensor;
determining at least one inertial measurement unit (IMU) output of the athlete:
comparing the ME and the IMU output of the athlete; and
based on comparing, determining a performance of the athlete.

2. The method of claim 1, further comprising:
determining a heart rate (HR) of the athlete by a wearable electrocardiogram (ECG) sensor carried by the athlete; and
comparing the HR and the IMU output of the athlete.

3. The method of claim 1, wherein the IMU output is an output of an accelerometer.

4. The method of claim 1, wherein the IMU output is an output of a global positioning system (GPS).

5. The method of claim 1, wherein the IMU output is an output of a gyroscope.

6. The method of claim 1, further comprising:
determining whether the athlete is efficient at least in part based on comparing the ME and the IMU output of the athlete.

7. The method of claim 2, further comprising:
determining whether the athlete is fatigued at least in part based on comparing the HR and the IMU output of the athlete.

8. The method of claim 2, further comprising:
determining whether the athlete is efficient at least in part based on comparing the HR and the IMU output of the athlete MR.

9. A system for monitoring athletic performance of an athlete, comprising:
athlete's clothing comprising one or more articles of clothing;
a wearable electromyography (EMG) sensor configured for determining a muscle effort (ME) of the athlete;
at least one wearable inertial measurement unit (IMU) sensor configured for monitoring an output of the athlete; and
a wearable controller attached with the athlete's clothing, the controller being configured to produce data based at least in part on an input from the ME and an input from the IMU sensor: wherein the wearable controller is configured for;
comparing the output of the ME sensor and the output of the IMU sensor; and
based on comparing, determine an athletic performance of the athlete.

10. The system of claim 8, wherein the controller includes a wireless interface configured to communicate with the EMG sensor and the IMU sensor.

11. The system of claim 8, wherein the IMU sensor is an accelerometer.

12. The system of claim 8, wherein the IMU sensor is a global positioning system (GPS).

13. The system of claim 8, wherein the IMU sensor is a gyroscope.

14. The system of claim 8, further comprising:
a wearable electrocardiogram (ECG) sensor attached with the athlete's clothing, the ECG being configured for monitoring a heart rate (HR) of the athlete.

15. The system of claim 8, wherein the athletic performance of the athlete is a fatigue.

16. The system of claim 8, wherein the athletic performance of the athlete is an efficiency of the athlete.
